(19) United States
(12) Patent Application Publication
(10) Pub. No.: US 2018/0296203 A1
(43) Pub. Date: Oct. 18, 2018

(54) SOFT-TISSUE RETRACTOR

(71) Applicant: 3M INNOVATIVE PROPERTIES COMPANY, SAINT PAUL, MN (US)

(72) Inventors: NICHOLAS R. POWLEY, SAINT PAUL, MN (US); MATTHEW T. SCHOLZ, WOODBURY, MN (US)

(73) Assignee: 3M INNOVATIVE PROPERTIES COMPANY, SAINT PAUL, MN (US)

(21) Appl. No.: 15/767,457

(22) PCT Filed: Oct. 13, 2016
(86) PCT No.: PCT/US2016/056791

§ 371 Date: Apr. 11, 2018

Related U.S. Application Data
(60) Provisional application No. 62/243,185, filed on Oct. 19, 2015.

(51) Int. Cl. A61B 17/02

(52) U.S. Cl. CPC A61B 17/0206; A61B 2017/00862

(57) ABSTRACT

The present disclosure provides a tissue retractor. The retractor includes first and a second tissue-support assemblies that are moveably coupled together. Each of the first and second tissue-support assemblies includes a flexible sheet extending between two horizontal members, each sheet comprising a face configured to contact tissue. The tissue support assemblies are configured such that, in use, the first and second sheets exert substantially all of the force against the tissue that is to be retracted.

### CROSS REFERENCE TO RELATED APPLICATIONS

This application claims priority to U.S. Provisional Patent Application No. 62/243,185, filed Oct. 19, 2015, the disclosure of which is incorporated by reference in its entirety herein.

### BACKGROUND

Surgical retractors are customarily used during major surgery to hold back the incision area to expose the area in which the surgeon operates. Abdominal surgery presents particular problems because of the presence of large soft organs, especially long lengths of intestines. The positioning during surgery of skin and internal organs is vitally important to the success of the surgery. The skin and/or organs can obscure the surgeon's vision if they are not held back by retractors. On the other hand, the tissue and organs themselves are fragile and damage to either may result in contamination of the field of surgery, greatly increasing the risk of post-operative infection or may result in other post-operative complications.

Surgical absorbent clothes or sponges, sometimes with the addition of hard metal retractors, have been used to hold and separate organs (e.g., intestines) from the point of surgery.

Although a variety of surgical retractors have been used, there remains a need for a tissue retractor that functions to expose (e.g., provide access to) underlying organs and tissue and, in addition, functions to preserve the vitality of the tissue that is being retracted.

### SUMMARY

The present disclosure generally relates to an article used to retract tissue (e.g., skin) in order to provide improved access to underlying tissue and/or organs. The inventive article can be used during surgery, for example, to provide improved exposure (e.g., access and visibility). Advantageously, the use of flexible sheet material according to the present disclosure to support the tissue that is being retracted can minimize pressure points on the retracted tissue in order to maintain perfusion of the retracted tissue during the surgical procedure. Maintenance of normal or near-normal perfusion can minimize tissue injury and promote faster healing after the surgical procedure. The articles of the present disclosure improve tissue perfusion over prior art retractors that provide relatively-inflexible tissue-support surfaces. The flexible sheet material may also be optically clear or translucent so that the retracted tissue can be monitored by visual assessment.

In one aspect, the present disclosure provides a tissue retractor. The tissue retractor can comprise a first tissue-support assembly and a second tissue-support assembly coupled to the first tissue-support assembly. The first tissue-support assembly can comprise a first longitudinal member having a first longitudinal end, a second longitudinal end opposite the first longitudinal end, and a first edge; a second longitudinal member having a third longitudinal end, a fourth longitudinal end opposite the third longitudinal end, and a second edge; a first lateral member extending between the first and second longitudinal members, the first lateral member having a first upper end attached to the first longitudinal member, a first lower end attached to the second longitudinal member, and a first indent region disposed between the first upper end and the first lower end; and a first sheet of pliable tissue-contact material coupled to the first tissue-support assembly, the first sheet extending from the first edge to the second edge. The second tissue-support assembly can comprise a third longitudinal member having a fifth end, a sixth end opposite the fifth end, and a third edge; a fourth longitudinal member having a seventh end, an eighth end opposite the seventh end, and a fourth edge; a third lateral member extending between the third and fourth longitudinal members, the third lateral member having a third upper end attached to the third longitudinal member, a third lower end attached to the fourth longitudinal member, and a third indent region disposed between the third upper end and the third lower end; and a second sheet of pliable tissue-contact material coupled to the second tissue-support assembly, the second sheet extending from the third edge to the fourth edge. The first sheet comprises a first face and a second face opposite the first face, wherein the first face faces away from the first indent region. The second sheet comprises a third face and a fourth face opposite the third face, wherein the third face faces away from the third indent region. The first face faces away from the third face. The first sheet does not contact the first indent region and the second sheet does not contact the third indent region.

In any of the above embodiments, the first tissue-support assembly further can comprise a second lateral member extending between the first and second longitudinal member. The second lateral member has a second upper end attached to the first longitudinal member, a second lower end attached to the second longitudinal member, and a second indent region disposed between the second upper end and the second lower end. The second lateral member is spaced apart from the first lateral member. The first sheet does not contact the second indent region.

In any of the above embodiments, the second tissue-support assembly further can comprise a fourth lateral member extending between the third and fourth longitudinal members. The fourth lateral member has a fourth upper end attached to the third longitudinal member, a fourth lower end attached to the fourth longitudinal member, and a fourth indent region disposed between the fourth upper end and the fourth lower end. The second sheet does not contact the fourth indent region.

In any of the above embodiments, the tissue retractor further can comprise a translating element that is adapted to move the first tissue-support assembly away from the second tissue-support assembly or to move the second tissue-support assembly away from the first tissue-support assembly.

In any of the above embodiments, the first sheet or the second sheet can comprise a plurality of layers.

In any of the above embodiments, the tissue retractor further can comprise a source of the electromagnetic radiation operatively coupled thereto.

The terms “comprises” and variations thereof do not have a limiting meaning where these terms appear in the description and claims.

As used herein, “a,” “an,” “the,” “at least one,” and “one or more” are used interchangeably. Thus, for example, a binding partner can be interpreted to mean “one or more” binding partners.

The term “and/or” means one or all of the listed elements or a combination of any two or more of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

Additional details of these and other embodiments are set forth in the accompanying drawings and the description below. Other features, objects and advantages will become apparent from the description and drawings, and from the claims.

### BRIEF DESCRIPTION OF DRAWINGS

FIG. 1 is a plan view of one embodiment of a tissue retractor according to the present disclosure.

FIG. 2 is an end view of the tissue retractor of FIG. 1.

FIG. 3 is one side view of the tissue retractor of FIG. 1, showing the tissue-facing side of the first sheet.

FIG. 4 is another side view of the tissue retractor of FIG. 1, showing the tissue-facing side of the second sheet.

FIG. 5A is a perspective view of the tissue retractor of FIG. 1.

FIG. 5B is another perspective view of the tissue retractor of FIG. 1.

FIG. 6A is a side view of the tissue retractor of FIG. 1, showing the first sheet comprises a first central axis.

FIG. 6B is a side view of the tissue retractor of FIG. 1, showing the second sheet comprises a second central axis.

FIG. 7A is a side view of one embodiment of a tissue retractor comprising a fenestrated first sheet.

FIG. 7B is a side view of another embodiment of a tissue retractor comprising a fenestrated first sheet, wherein at least one fenestration extends from the first edge to the second edge of the first tissue-support assembly.

FIG. 8A is a plan view of one embodiment of a first sheet comprising a plurality of layers.

FIG. 8B is an exploded side view of the first sheet of FIG. 8A.

FIG. 9A is a plan view of one embodiment of a second sheet comprising a plurality of layers.

FIG. 9B is an exploded side view of the second sheet of FIG. 9A.

FIGS. 10A-D are end views, partially in section, showing various steps of one embodiment of a method of retracting tissue at a wound site according to the present disclosure.

### DETAILED DESCRIPTION

Before any embodiments of the present disclosure are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “connected” and “coupled” and variations thereof are used broadly and encompass both direct and indirect connections and couplings. Further, “connected” and “coupled” are not restricted to physical or mechanical connections or couplings. It is to be understood that other embodiments may be utilized and structural or logical changes may be made without departing from the scope of the present disclosure. Furthermore, terms such as “front,” “rear,” “top,” “bottom,” and the like are only used to describe elements as they relate to one another, but are in no way meant to recite specific orientations of the device, to indicate or imply necessary or required orientations of the device, or to specify how the invention described herein will be used, mounted, displayed, or positioned in use.

The present disclosure generally relates to surgical retractors and instruments that, where applicable, maximize exposure, tissue traction, and tissue health of target and peripheral tissue. The surgical retractors and instruments able to maximize tissue health while yielding maximal exposure and tissue traction of both (1) soft and delicate tissues such as nerves, blood vessels, and other sensitive anatomy, and (2) more robust tissues such as the epidermis and fat layers, ribs, and other bones.

Generally, each retractor or instrument of the present disclosure is able to apply a force to a target tissue or anatomical structure via two or more sheets of pliable tissue-contact material, the sheets being supported by a plurality of tissue-support assemblies that are optionally are controlled by a translating element and/or a position controller as described herein. Specifically, the retractor or instrument is adapted to apply a force to at least one target (e.g., a tissue or tissues, an organ or organs) within or extending from an area of action defined by a portion of the tissue-contact material, without requiring direct contact between the target and one or more of the tissue-support assemblies. Advantageously, a surgical retractor or instrument of the present disclosure is capable of moving and/or retaining the position of a tissue or organ without exerting excess pressure to the tissue and/or organ, thereby maximizing post-operative health of the tissue or organ.

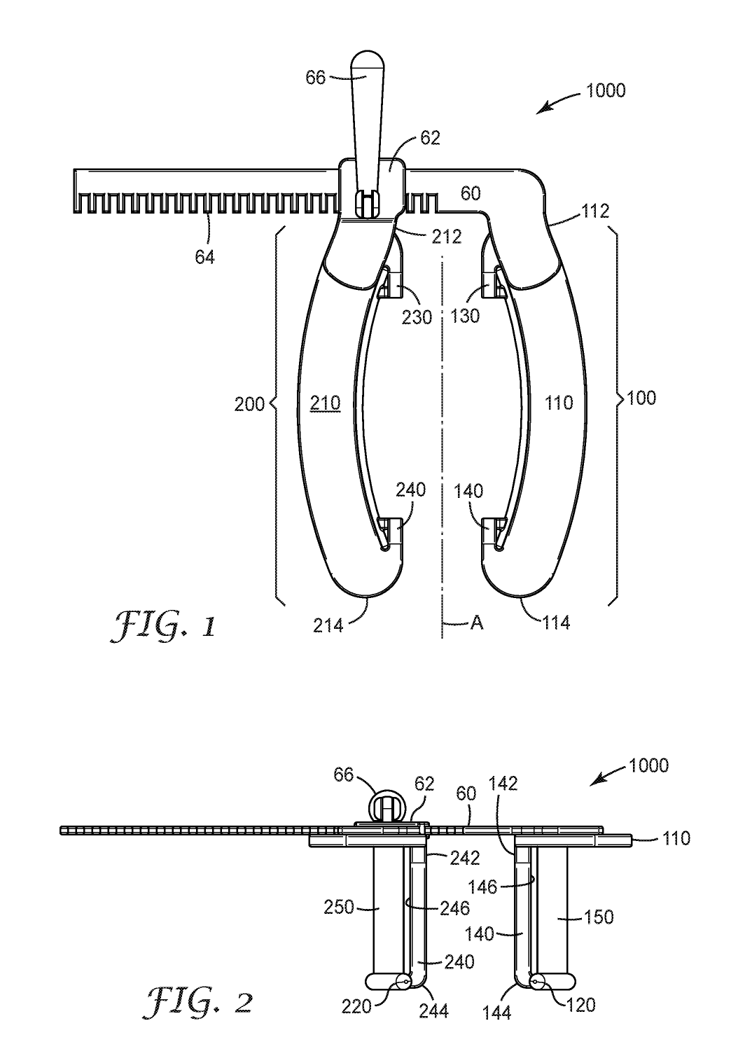

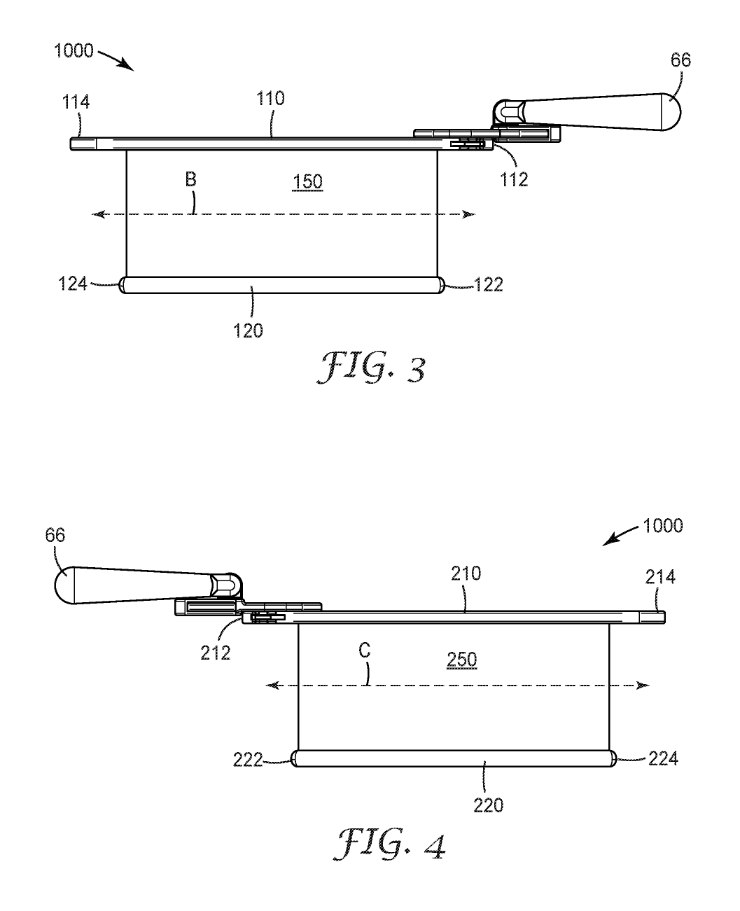

Turning to the drawings, FIGS. 1-5B show various views of one embodiment of a tissue retractor 1000 according to the present disclosure. The tissue retractor 1000 comprises a plurality of tissue-support assemblies including a first tissue-support assembly 100 and a second tissue-support assembly 200. Each tissue-support assembly comprises a plurality of structural elements that form a frame or scaffold that supports a tissue-contact material. The structural elements of the frame are relatively rigid compared to the tissue-contact material, as discussed herein.

The first tissue-support assembly 100 comprises a first longitudinal member 110.

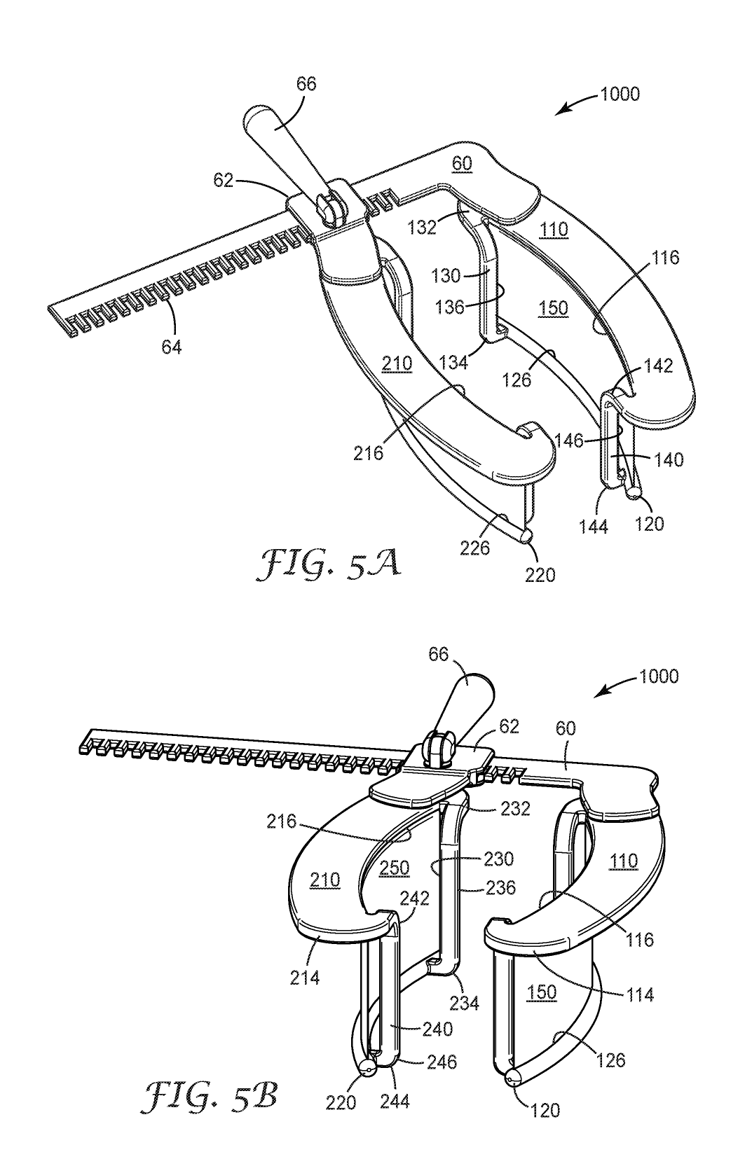

The first longitudinal member 110 has a first longitudinal end 112, a second longitudinal end 114 opposite the first longitudinal end, and a first edge 116 disposed between the first longitudinal end and second longitudinal end. In any embodiment, the first edge 116 is generally flat (e.g., not serrated or scalloped), smooth (e.g., without sharp projections or a cutting surface), and defines a straight linear (not shown) or curvilinear line extending along the first longitudinal member 110 between the first longitudinal end 112 and the second longitudinal end 114. In any embodiment, the first edge 116 may define a contoured (e.g., wavy) line extending along the first longitudinal member 110 between the first longitudinal end 112 and the second longitudinal end 114. In addition, the first edge 116 may be curved, as shown in FIGS. 1 and 5A, or straight (not shown). Although illustrated as a plate-like structure having length, width, and depth (thickness) dimensions wherein the width dimension is greater than the depth dimension, it is contemplated that the first longitudinal member 110 can take a variety of forms including, but not limited to, a cylindrical rod (not shown).

The first tissue-support assembly 100 further comprises a second longitudinal member 120. The second longitudinal member 120 has a third longitudinal end 122, a fourth longitudinal end 124 opposite the third longitudinal end, and a second edge 126 disposed between the third longitudinal end and fourth longitudinal end. In any embodiment, the second edge 126 is generally flat (e.g., not serrated or scalloped) and smooth (e.g., without sharp projections or a cutting surface). In addition, the second edge 126 may be curved, as shown in FIGS. 1 and 5A, or straight (not shown). Although illustrated as a cylindrical rod-like structure having length, width, and depth (diameter) dimensions, it is contemplated that the second longitudinal member 120 can take a variety of forms including, but not limited to, a plate-like form similar to that of the first longitudinal member 110.

Extending between and coupled (e.g., directly or indirectly coupled) to the first longitudinal member 110 and the second longitudinal member 120 is a first lateral member 130. The first lateral member 130 has a first upper end 132 attached (e.g., either directly or indirectly) to the first longitudinal member 110, a first lower end 134 attached (e.g., either directly or indirectly) to the second longitudinal member 120, and a first indent region 136 disposed between the first upper end and the first lower end. In any embodiment, the first upper end 132 is attached to the first longitudinal member 110 proximate the first longitudinal end 112 and the first lower end 134 is attached to the second longitudinal member 120 proximate the third longitudinal end 122, as shown in FIGS. 1 and 5A. Alternatively, in any embodiment, the first upper end is attached to the first longitudinal member proximate the second longitudinal end and the first lower end is attached to the second longitudinal member proximate the fourth longitudinal end (not shown). Alternatively, in any embodiment, the first upper end is attached to the first longitudinal member at a location between (e.g., about half-way between) the first longitudinal end and the second longitudinal end and the first lower end is attached to the second longitudinal member at a location between (e.g., about half-way between) the third longitudinal end and the fourth longitudinal end (not shown).

Although illustrated as a plate-like structure having length, width, and depth (thickness) dimensions wherein the width dimension is greater than the depth dimension, it is contemplated that the first lateral member 130 can take a variety of forms including, but not limited to, a cylindrical rod (not shown).

In the illustrated embodiment of FIGS. 1-5B, the first longitudinal member 110 and first lateral member 130 are formed of the same material (e.g., metal, plastic) as a unitary part. However, it is contemplated the first longitudinal member 110 and first lateral member 130 may be formed separately, optionally from different materials, and subsequently joined together using attachment means (e.g., fasteners, clamps, adhesives, welds, or the like) that are known in the art.

Examples of suitable materials from which the longitudinal and lateral members can be made include 300 series and 400 series stainless steel (e.g., 304 and 316 stainless steel), acrylics (e.g., polymethylmethacrylate), polysulfone, polyetherimides (e.g., ULTEM® PEI plastics), as well as composites of thermoplastic or thermoset polymers including glass and carbon fiber epoxy and polyurethane composites. Preferred materials are sterilizable by heat (e.g., steam sterilization at 120-135° C.). Other methods of sterilization may include gamma radiation and relatively low-temperature (<60° C.) sterilization using ethylene oxide, hydrogen peroxide or peracetic acid.

In the illustrated embodiment of FIGS. 1-5B, the second longitudinal member 120 and first lateral member 130 are formed as separate parts, optionally from different materials, and are subsequently joined together using attachment means (e.g., fasteners, clamps, adhesives, welds, or the like) that are known in the art. However, it is contemplated that the second longitudinal member 120 and first lateral member 130 may be formed together as a unitary part, the unitary part optionally including the first longitudinal member 110.

Optionally, the first tissue-support assembly 100 comprises a second lateral member 140. The second lateral member 140 extends between and is coupled (e.g., directly or indirectly coupled) to the first longitudinal member 110 and the second longitudinal member 120. The second lateral member 140 has a second upper end 142 attached (e.g., either directly or indirectly) to the first longitudinal member 110, a second lower end 144 attached (e.g., either directly or indirectly) to the second longitudinal member 120, and a second indent region 146 disposed between the second upper end and the second lower end. In any embodiment, the second upper end 142 may be attached to the first longitudinal member 110 proximate the second longitudinal end 114 and the second lower end 144 may be attached to the second longitudinal member 120 proximate the fourth longitudinal end 124, as shown in the illustrated embodiment. Alternatively, in any embodiment, the second upper end is attached to the first longitudinal member at a location between (e.g., about half-way between) the first longitudinal end and the second longitudinal end and the second lower end is attached to the second longitudinal member at a location between (e.g., about half-way between) the third longitudinal end and the fourth longitudinal end (not shown).

Although illustrated as a plate-like structure having length, width, and depth (thickness) dimensions wherein the width dimension is greater than the depth dimension, it is contemplated that the second lateral member 140 can take a variety of forms including, but not limited to, a cylindrical rod (not shown).

In the illustrated embodiment of FIGS. 1-5B, the first longitudinal member 110 and the optional second lateral member 140 are formed of the same material (e.g., metal, plastic, or composites, as described above) as a unitary part. However, it is contemplated the first longitudinal member 110 and second lateral member 140 may be formed separately, optionally from different materials, and subsequently joined together using attachment means (e.g., fasteners, clamps, adhesives, welds, or the like) that are known in the art.

In the illustrated embodiment of FIGS. 1-5B, the second longitudinal member 120 and second lateral member 140 are formed as separate parts, optionally from different materials, and are subsequently joined together using attachment means (e.g., fasteners, clamps, adhesives, welds, or the like) that are known in the art. However, it is contemplated that the second longitudinal member 120 and second lateral member 140 may be formed together as a unitary part, the unitary part optionally including the first longitudinal member 110 and the first lateral member 130.

Coupled to the first tissue-support assembly 100 and extending between the first edge 116 and second edge 126 is a first sheet 150 of tissue-contact material. The first sheet 150 comprises a pliable, sheet-like material (e.g., a polymeric film, a nonwoven fabric material, a woven fabric material, a knit material, a porous film (e.g., a membrane or a foam sheet) or a combination of any two or more of the foregoing materials including thermal and adhesive-bonded laminates) that can come into contact with live biological tissue without causing a substantial adverse effect on the tissue. Preferably, the first sheet 150 is fabricated from one or more materials that is not substantially degraded when exposed to a disinfection and/or decontamination process (e.g., a heat, chemical, or electromagnetic irradiation process). In any embodiment, the first sheet 150 may comprise a light-guiding polymeric film such as, for example, the light-guiding films described in U.S. Pat. Nos. 9,070,312 and 9,039,245. In any embodiment, the first sheet 150 may comprise an optically-diffusing (e.g., light-scattering) polymeric film. Diffuse light can be provided, for example, by providing a texture on the wound-facing surface of the sheet. In any embodiment, the texture can be a random matte finish or an engineered microreplicated pattern. In these embodiments, it is preferable the side of the sheet that receives the light has a low refractive index. This can be provided by fluoropolymer films, for example. In any embodiment, the first sheet 150 may comprise a light-transmissive, optically clear or translucent polymeric film.

In any embodiment, the first sheet can comprise an engineered film such as, for example, a fluid-control film (e.g., fluid-transport film) disclosed in U.S. Pat. No. 6,290,685, which is incorporated herein by reference in its entirety.

Generally, the pliable material of the first sheet 150 is extended between the first edge 116 and the second edge 126 in a manner that does not permit the first sheet to contact the first lateral member 130 at the first indent region 136 or the second lateral member 140, if present, at the second indent region 146. In certain embodiments, the pliable material of the first sheet 150 extending between the first edge 116 and the second edge 126 does not contact any part of the first lateral member 130 or any part of the second lateral member 140, if present. This configuration advantageously causes the tissue (e.g., skin, subdermal connective tissue or adipose tissue) being retracted by the tissue retractor 1000 to have contact distributed over the relatively large, pliable material of the first sheet 150 rather than the relatively small, less flexible material of any of the components of the first tissue-support assembly 100; thereby minimizing the possibility of tissue contact with relatively small, nonpliable components that could cause pressure points that may hinder local blood circulation while the tissue is retracted. Advantageously, a plastic film could distribute force against the tissue better (e.g., more uniformly and/or over a larger surface area and/or with fewer or smaller pressure points) than a retractor having metal or hard-plastic tissue contact face.

The first sheet 150 has a first face and a second face opposite the first face. The first face faces away from the first indent region 136 and the second face faces toward the first indent region. Thus, the first face faces toward the tissue that is supported/retracted by the tissue retractor 1000.

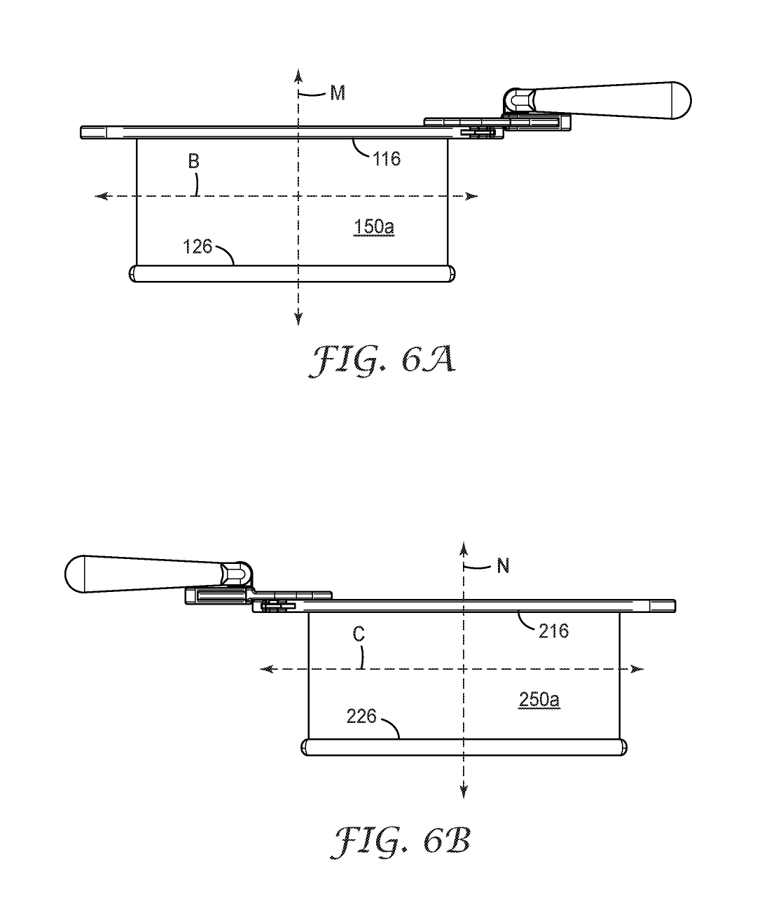

In any embodiment, the first sheet 150 comprises a tissue-support portion, as shown in FIG. 6A. The first tissue-support portion 150a does not overlap the first longitudinal member or the second longitudinal member. In any embodiment, the first sheet further may comprise another portion (not shown) that overlaps at least a part (e.g., the first edge116) of the first longitudinal member 110 and is secured (e.g., via an adhesive and/or a clamping means) to the first longitudinal member. Additionally or alternatively, in any embodiment, the first sheet further can comprise another portion (not shown) that overlaps at least a part (e.g., the second edge 126) of the second longitudinal member 120. The first tissue-support portion 150a extends between the first edge 116 of the first longitudinal member 110 and the second edge 126 of the second longitudinal member 120 and does not overlap either a part of the first longitudinal member or a part of the second longitudinal member. In use, the first tissue-support portion 150a contacts the tissue (not shown) that is being retracted by the tissue retractor of the present disclosure. Advantageously, this configuration minimizes the possibility of tissue contact with relatively small, nonpliable components of a tissue retractor that could cause pressure points that may hinder local blood circulation while the tissue is retracted.

In any embodiment, the first tissue-support portion 150a of the first sheet 150 forms a plane. In any embodiment, the plane formed by the first tissue-support portion 150a can be a flat plane (as illustrated in FIGS. 1-5B) or a curved plane (not shown). In any embodiment, the plane formed by the first tissue-support portion 150a can be a continuous plane (i.e., the plane is unbroken between any of the edges) or it can be a fenestrated plane (i.e., the first sheet 150 includes one or more openings or fenestrations). In any embodiment, one or more of the fenestrations can extend from the first edge 116 to the second edge 126.

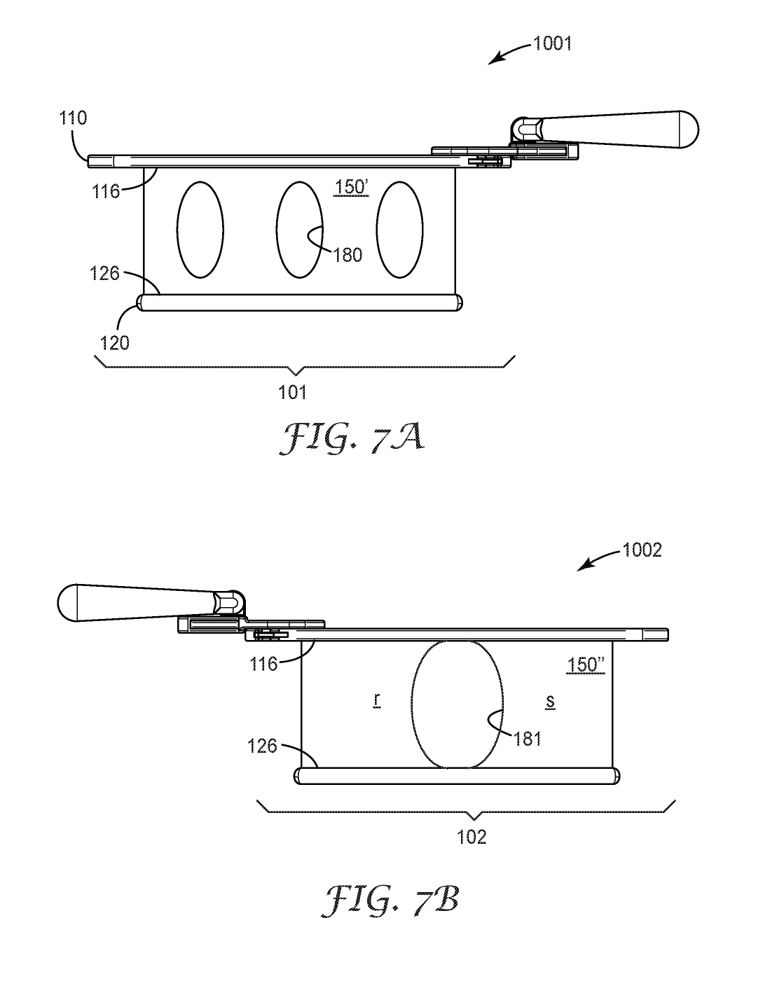

FIG. 7A shows one embodiment of a tissue retractor 1001 that includes a first tissue-support assembly 101 comprising a first sheet 150′ that extends from the first edge 116 of the first longitudinal member 110 to the second edge 126 of the second longitudinal member. The first sheet 150′ includes a plurality of fenestrations 180. Although the fenestrations 180 are illustrated as having an oval shape, it is contemplated that the fenestrations may have any geometric shape including, for example, a circle, a rectangle, a triangle, or a square. FIG. 7B shows one alternative embodiment of a tissue retractor 1002 that includes a first tissue-support assembly 102 comprising a first sheet 150″ that includes a fenestration 181 that extends from the first edge 116 of the first longitudinal member 110 to the second edge 126 of the second longitudinal member 120. Thus the fenestration 181 extending from the first edge 116 to the second edge 126 effectively splits the tissue-contact portion of the first sheet 150″ into two distinct spaced-apart sections “r” and “s”, respectively. Advantageously, these spaced-apart sections can independently support different types (or portions of the same type) of tissue, thereby providing better conformance to the anatomical site in which the retractor is used. It is contemplated that any embodiment of a tissue retractor of the present disclosure could additionally, or alternatively, comprise a second sheet that has one or more fenestration as described herein.

A tissue retractor of the present disclosure has a first longitudinal axis “A” extending generally in the direction of the first tissue-support assembly 100 and the second tissue-support assembly 200, as shown in FIG. 1. In addition, the first sheet 150 comprises a first sheet central axis “B” that extends substantially parallel to the first longitudinal axis “A”. The first sheet 150 has a first sheet tensile strength. When a force that is less than the first sheet tensile strength is applied to the first sheet central axis, the first sheet does not contact the first indent region and the first sheet does not contact the second indent region, if present.

The first sheet 150 has a first elastic modulus of about 1 kPa to about 20 GPa; preferably, about 0.1-20 GPa; along a first reference axis “M” that is orthogonal to the first sheet central axis “B”. A person having ordinary skill in the art will appreciate the elastic modulus of the first sheet used in any embodiment of a tissue retractor according to the present disclosure will be selected at least in accordance with the depth of the indents, taking care that the first sheet, when subject to force from the tissue being retracted, will not stretch enough to come into contact with the first and/or second indents.

The second tissue-support assembly 200 comprises a third longitudinal member 210. The third longitudinal member 210 has a fifth longitudinal end 212, a sixth longitudinal end 214 opposite the fifth longitudinal end, and a third edge 216 disposed between the third longitudinal end and fourth longitudinal end. In any embodiment, the third edge 216 is generally flat (e.g., not serrated or scalloped), smooth (e.g., without sharp projections or a cutting surface), and defines a straight linear (not shown) or curvilinear line extending along the third longitudinal member 210 between the fifth longitudinal end 212 and the sixth longitudinal end 214. In any embodiment, the third edge 216 may define a contoured (e.g., wavy) line extending along the third longitudinal member 210 between the fifth longitudinal end 212 and the sixth longitudinal end 214. In addition, the third edge 216 may be curved, as shown in FIGS. 1 and 5B, or straight (not shown). Although illustrated as a plate-like structure having length, width, and depth (thickness) dimensions wherein the width dimension is greater than the depth dimension, it is contemplated that the third longitudinal member 210 can take a variety of forms including, but not limited to, a cylindrical rod (not shown).

The second tissue-support assembly 200 further comprises a fourth longitudinal member 220. The fourth longitudinal member 220 has a seventh longitudinal end 222, an eighth longitudinal end 224 opposite the seventh longitudinal end, and a fourth edge 226 disposed between the seventh longitudinal end and eighth longitudinal end. In any embodiment, the fourth edge 226 is generally flat (e.g., not serrated or scalloped) and smooth (e.g., without sharp projections or a cutting surface). In addition, the fourth edge 226 may be curved, as shown in FIGS. 1 and 5B, or straight (not shown). Although illustrated as a cylindrical rod-like structure having length, width, and depth (diameter) dimensions, it is contemplated that the fourth longitudinal member 220 can take a variety of forms including, but not limited to, a plate-like form similar to that of the third longitudinal member 210.

Extending between and coupled (e.g., directly or indirectly coupled) to the third longitudinal member 210 and the fourth longitudinal member 220 is a third lateral member 230. The third lateral member 230 has a third upper end 232 attached (e.g., either directly or indirectly) to the third longitudinal member 210, a third lower end 234 attached (e.g., either directly or indirectly) to the fourth longitudinal member 220, and a third indent region 236 disposed between the third upper end and the third lower end. In any embodiment, the third upper end 232 is attached to the third longitudinal member 210 proximate the fifth longitudinal end 212 and the third lower end 234 is attached to the fourth longitudinal member 220 proximate the seventh longitudinal end 222, as shown in FIGS. 1 and 5B. Alternatively, in any embodiment, the third upper end is attached to the third longitudinal member proximate the sixth longitudinal end and the third lower end is attached to the fourth longitudinal member proximate the eighth longitudinal end (not shown). Alternatively, in any embodiment, the third upper end is attached to the third longitudinal member at a location between (e.g., about half-way between) the fifth longitudinal end and the sixth longitudinal end and the third lower end is attached to the fourth longitudinal member at a location between (e.g., about half-way between) the seventh longitudinal end and the eighth longitudinal end (not shown).

Although illustrated as a plate-like structure having length, width, and depth (thickness) dimensions wherein the width dimension is greater than the depth dimension, it is contemplated that the third lateral member 230 can take a variety of forms including, but not limited to, a cylindrical rod (not shown).

In the illustrated embodiment of FIGS. 1-5B, the third longitudinal member 210 and third lateral member 230 are formed of the same material (e.g., metal, plastic) as a unitary part. However, it is contemplated the third longitudinal member 210 and third lateral member 230 may be formed separately, optionally from different materials, and subsequently joined together using attachment means (e.g., fasteners, clamps, adhesives, welds, or the like) that are known in the art.

Examples of suitable materials from which the longitudinal and lateral members can be made include 300 series and 400 series stainless steel (e.g., 304 and 316 stainless steel), acrylics (e.g., polymethylmethacrylate), polysulfone, polyetherimides (e.g., ULTEM® PEI plastics), as well as composites of thermoplastic or thermoset polymers including glass and carbon fiber epoxy and polyurethane composites. Preferred materials are sterilizable by heat (e.g., steam sterilization at 120-135° C.). Other methods of sterilization may include gamma radiation and relatively low-temperature (<60° C.) sterilization using ethylene oxide, hydrogen peroxide or peracetic acid.

In the illustrated embodiment of FIGS. 1-5B, the fourth longitudinal member 220 and third lateral member 230 are formed as separate parts, optionally from different materials, and are subsequently joined together using attachment means (e.g., fasteners, clamps, adhesives, welds, or the like) that are known in the art. However, it is contemplated that the third longitudinal member 210 and third lateral member 230 may be formed together as a unitary part, the unitary part optionally including the third longitudinal member 210.

Optionally, the second tissue-support assembly 200 comprises a fourth lateral member 240. The fourth lateral member 240 extends between and is coupled (e.g., directly or indirectly coupled) to the third longitudinal member 210 and the fourth longitudinal member 220. The fourth lateral member 240 has a fourth upper end 242 attached (e.g., either directly or indirectly) to the third longitudinal member 210, a fourth lower end 244 attached (e.g., either directly or indirectly) to the fourth longitudinal member 220, and a fourth indent region 246 disposed between the fourth upper end and the fourth lower end. In any embodiment, the fourth upper end 242 may be attached to the third longitudinal member 210 proximate the sixth longitudinal end 214 and the fourth lower end 244 may be attached to the fourth longitudinal member 220 proximate the eighth longitudinal end 224, as shown in the illustrated embodiment. Alternatively, in any embodiment, the fourth upper end is attached to the third longitudinal member at a location between (e.g., about half-way between) the fifth longitudinal end and the sixth longitudinal end and the fourth lower end is attached to the fourth longitudinal member at a location between (e.g., about half-way between) the seventh longitudinal end and the eighth longitudinal end (not shown).

Although illustrated as a plate-like structure having length, width, and depth (thickness) dimensions wherein the width dimension is greater than the depth dimension, it is contemplated that the fourth lateral member 240 can take a variety of forms including, but not limited to, a cylindrical rod (not shown).

In the illustrated embodiment of FIGS. 1-5B, the third longitudinal member 210 and the optional fourth lateral member 240 are formed of the same material (e.g., metal, plastic) as a unitary part. However, it is contemplated the third longitudinal member 210 and fourth lateral member 240 may be formed separately, optionally from different materials, and subsequently joined together using attachment means (e.g., fasteners, clamps, adhesives, welds, or the like) that are known in the art.

In the illustrated embodiment of FIGS. 1-5B, the fourth longitudinal member 220 and fourth lateral member 240 are formed as separate parts, optionally from different materials, and are subsequently joined together using attachment means (e.g., fasteners, clamps, adhesives, welds, or the like) that are known in the art. However, it is contemplated that the fourth longitudinal member 220 and fourth lateral member 240 may be formed together as a unitary part, the unitary part optionally including the third longitudinal member 210 and the third lateral member 230.

Coupled to the second tissue-support assembly 200 and extending between the third edge 216 and fourth edge 226 is a second sheet 250 of tissue-contact material. The second sheet 250 comprises a pliable, sheet-like material (e.g., a polymeric film, a nonwoven fabric material, a woven fabric material, a knit material, a porous film (e.g., a membrane or a foam sheet) or a combination of any two or more of the foregoing materials including thermal and adhesive-bonded laminates) that can come into contact with live biological tissue without causing a substantial adverse effect on the tissue or a combination of any two or more of the foregoing materials) that can come into contact with live biological tissue without causing a substantial adverse effect on the tissue. Preferably, the second sheet 250 is fabricated from one or more materials that is not substantially degraded when exposed to a disinfection and/or decontamination process (e.g., a heat, chemical, or electromagnetic irradiation process). In any embodiment, the second sheet 250 may comprise a light-guiding polymeric film such as, for example, the light-guiding films described in U.S. Pat. Nos. 9,070,312 and 9,039,245. In any embodiment, the second sheet 250 may comprise an optically-diffusing (e.g., light-scattering) polymeric film. Diffuse light can be provided, for example, by providing a texture on the wound-facing surface of the sheet. In any embodiment, the texture can be a random matte finish or an engineered microreplicated pattern. In these embodiments, it is preferable the side of the sheet that receives the light has a low refractive index. This can be provided by fluoropolymer films, for example. In any embodiment, the second sheet 250 may comprise a light-transmissive, optically clear or translucent polymeric film.

In any embodiment, the second sheet can comprise an engineered film such as, for example, a fluid-control film (e.g., fluid-transport film) disclosed in U.S. Pat. No. 6,290,685, which is incorporated herein by reference in its entirety.

Generally, the pliable material of the second sheet 250 is extended between the third edge 216 and the fourth edge 226 in a manner that does not permit the second sheet to contact the third lateral member 230 at the third indent region 236 or the fourth lateral member 240, if present, at the fourth indent region 246. In certain embodiments, the pliable material of the second sheet 250 extending between the third edge 216 and the fourth edge 226 does not contact any portion of the third lateral member 230 or any portion of the fourth lateral member 240, if present. This configuration advantageously causes the tissue (e.g., skin, subdermal connective tissue or adipose tissue) being retracted by the tissue retractor 1000 to have contact distributed over the relatively large, pliable material of the second sheet 250 rather than the relatively small, less flexible material of any of the components of the second tissue-support assembly 200; thereby minimizing the possibility of tissue contact with relatively small, nonpliable components that could cause pressure points that may hinder local blood circulation while the tissue is retracted.

The second sheet 250 has a third face and a fourth face opposite the third face. The third face faces away from the third indent region 236 and the fourth face faces toward the third indent region 236. Thus, the third face faces toward the tissue that is supported/retracted by the tissue retractor 1000.

In any embodiment, the second sheet 250 comprises a second tissue-support portion, as shown in FIG. 6B. The second tissue-support portion 250a does not overlap the first longitudinal member or the second longitudinal member. In any embodiment, the second sheet 250 further may comprise another portion (not shown) that overlaps at least a part (e.g., the third edge 216) of the third longitudinal member 210 and is secured (e.g., via an adhesive and/or a clamping means) to the third longitudinal member. Additionally or alternatively, in any embodiment, the second sheet 250 further can comprise another portion (not shown) that overlaps at least a part (e.g., the third edge 216) of the third longitudinal member 210. The second tissue-support portion 250a extends between the third edge 216 of the third longitudinal member 210 and the fourth edge 226 of the fourth longitudinal member 220 and does not overlap either a part of the third longitudinal member or a part of the fourth longitudinal member. In use, the second tissue-support portion 250a contacts the tissue (not shown) that is being retracted by the tissue retractor of the present disclosure. Advantageously, this configuration minimizes the possibility of tissue contact with relatively small, nonpliable components of a tissue retractor that could cause pressure points that may hinder local blood circulation while the tissue is retracted.

In any embodiment, the second tissue-support portion 250a of the second sheet 250 forms a plane. In any embodiment, the plane formed by the second tissue-support portion 250a can be a flat plane (as illustrated in FIGS. 1-51-5B) or a curved plane (not shown). In any embodiment, the plane formed by the second tissue-support portion 250a can be a continuous plane (i.e., the plane is unbroken between any of the edges) or it can be a fenestrated plane (i.e., the second sheet 250 includes openings or fenestrations, not shown).

A tissue retractor of the present disclosure has a longitudinal axis “A” extending generally in the direction of the first tissue-support assembly 100 and the second tissue-support assembly 200, as shown in FIG. 1. In addition, the second sheet 250 comprises a second sheet central axis “C” that extends substantially parallel to the longitudinal axis “A”. The second sheet 250 has a second sheet tensile strength. When a force that is less than the second sheet tensile strength is applied to the second sheet central axis, the second sheet does not contact the third indent region and the second sheet does not contact the fourth indent region, if present.

The second sheet 250 has a first elastic modulus of about 1 kPa to about 20 GPa; preferably, about 0.1-20 GPa; along a first reference axis “N” that is orthogonal to the second sheet central axis “C”. A person having ordinary skill in the art will appreciate the elastic modulus of the second sheet used in any embodiment of a tissue retractor according to the present disclosure will be selected at least in accordance with the depth of the indents, taking care that the second sheet, when subject to force from the tissue being retracted, will not stretch enough to come into contact with the third and/or fourth indents.

In addition, the first sheet 150 and the second sheet 250 both have a strain limit that defines a maximum force they each can withstand without breaking. A person having ordinary skill in the art will recognize the strain limit depends upon parameters such as, for example, the material and the thickness of the first sheet 150 and the second sheet 250. The parameters guide the fabrication of sheets 150 and 250 such that they are capable of withstanding enough force to retract the skin at a wound and/or retract an organ in a wound site without permitting undue force that could lead to tissue and/or organ damage (e.g., due to crushing or circulatory impairment). In any embodiment of a retractor according to the present disclosure, the first sheet 150, and the second sheet 250, each have a strain limit of about 2 MPa to about 2 GPa. In any embodiment of a retractor according to the present disclosure the strain limit of the first sheet 150 may be the same or different from the strain limit of the second sheet 250.

The first tissue-support assembly 100 is coupled to the second tissue-support assembly 200. Preferably, the first tissue-support assembly 100 is movably coupled to the second tissue-support assembly 200. For example, in the illustrated embodiment of FIGS. 1-5B, the tissue retractor 1000 comprises a positioning element 60 that is fixedly attached to one of the tissue-support assemblies (e.g., first tissue-support assembly 100 as shown in FIG. 1) and a position controller 62 that is fixedly attached to the other tissue-support assembly (e.g., second tissue-support assembly 200 as shown in FIG. 1). The positioning element 60 can comprise position indexing structures 64 (e.g., gear teeth or the like) that function to position the tissue-support assemblies at predefined positions relative to each other. The position controller 62 is movably engaged (e.g., via the position indexing structures 64) with the positioning element 60, thus making the first tissue-support assembly 100 movably attached to the second tissue-support assembly 200. In some embodiments, the first tissue support assembly and/or second tissue support assembly can be detachably attached to the positioning element.

In any embodiment, the tissue retractor 1000 optionally can comprise a translating element 66 (e.g., a crank handle) that is adapted to work cooperatively with the positioning element 60 to move the first and second tissue support assemblies relative to each other. In any embodiment, the translating element 66 can move the first tissue-support assembly 100 away from the second tissue-support assembly 200. Additionally, or alternatively, the translating element 66 can move the second tissue-support assembly 200 away from the first tissue-support assembly 100.

In any embodiment, the translating element 66 can move the first tissue-support assembly 100 toward the second tissue-support assembly 200. Additionally, or alternatively, the translating element 66 can move the second tissue-support assembly 200 toward the first tissue-support assembly 100.

In any embodiment (not shown), a tissue retractor according to the present disclosure can comprise a pivot (e.g., the translating element comprises a pivot) at which the first and second tissue support assemblies are moveably connected. In these embodiments, the translating element (e.g., comprising a crank handle) is adapted to work cooperatively with the pivot to move the first and second tissue support assemblies relative to each other. The translating element can move the first and second tissue support assemblies relative to each other by moving the first tissue-support assembly away from the second tissue-support assembly. Additionally, or alternatively, the translating element can move the first and second tissue support assemblies relative to each other by moving the second tissue-support assembly away from the first tissue-support assembly.

In any embodiment of the tissue retractor of the present disclosure, the first sheet and/or the second sheet optionally may comprise a plurality of layers. Wherein the first sheet or second sheet comprises a plurality of layers, the plurality includes at least a tissue-contact layer and a substrate layer. The tissue-contact layer is disposed on the first face of the sheet and is intended to be in contact with the tissue supported by the tissue retractor. The substrate layer generally is disposed on the side of the tissue-contact layer that is opposite the tissue-facing side of the tissue contact layer. In any embodiment, the substrate layer may be disposed on the second face of the sheet.

In any embodiment, two or more of the plurality of layers may be coextensive. Alternatively, in any embodiment, one of the plurality of layers (e.g., the tissue contact layer or the substrate layer) may have a larger area than one or more of the other layer(s) of the plurality.

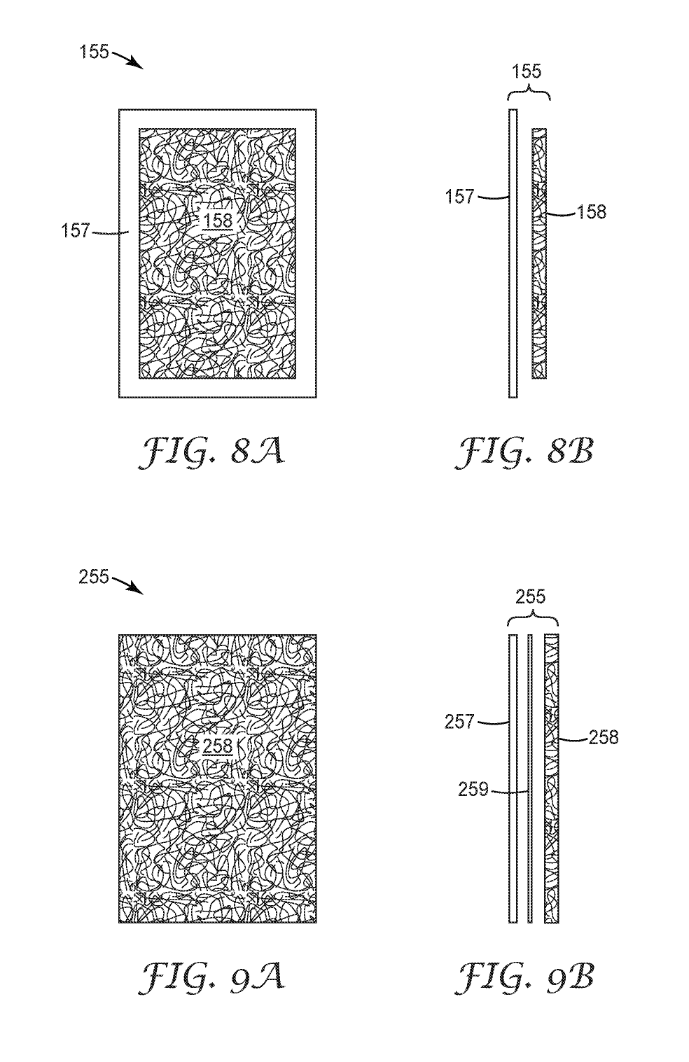

FIGS. 8A and 8B shows separate views of one embodiment of a first sheet 155 that comprises a plurality of layers. The plurality comprises a first substrate layer 157 and a first tissue-contact layer 158. In any embodiment, the first substrate layer 157 can comprise a polymeric film. Suitable films include multilayer films (e.g., coextruded films) and/or a film coated with a solvent solution polymer or polymer dispersion. Suitable polymers include polyolefins, polyesters, polyamides, polyurethanes, acrylic block and random copolymers, polyether amides, polyether polyesters, polyolefinpolyacrylate copolymers such as ethylenemethylacrylate, polyolefin copolymers with polar vinyl monomers such as polyethylene vinyl acetate and the like. The film preferably is formed from a transparent or translucent polymeric material. The material preferably permits moisture evaporation through the film during prolonged surgeries. Suitable materials include polyolefins, such as low density polyethylene and particularly metallocene polyethylenes such as ENGAGETM polyethylenes commercially available from Dow Chemical, polyurethanes such as polyester or polyether polyurethanes (e.g., “ESTANE™ thermoplastic polyurethane,” commercially available from B. F. Goodrich, Cleveland Ohio), polyesters such as polyether polyester (e.g., “HYTREL™ polyester elastomer,” commercially available from Du Pont Co., Wilmington, Del.), and polyamides such as polyether polyamides (e.g., “PEBAX™ Resins” commercially available from ELF Atochem, North America, Inc., Philadelphia, Pa.).

The first substrate layer 157, when present, functions to provide the tensile strength necessary to retract tissue when the first sheet is urged against the tissue. In any embodiment, the first substrate layer 157 may comprise a light-guiding polymeric film such as, for example, the light-guiding films described in U.S. Pat. Nos. 9,070,312 and 9,039,245. In any embodiment, the second sheet 250 may comprise an optically-diffusing (e.g., light-scattering) polymeric film. Diffuse light can be provided, for example, by providing a texture on the wound-facing surface of the sheet. In any embodiment, the texture can be a random matte finish or an engineered microreplicated pattern. In these embodiments, it is preferable the side of the sheet that receives the light has a low refractive index. This can be provided by fluoropolymer films, for example.

The first tissue-contact layer 158, when present, provides a biocompatible surface for contacting tissue in a wound (e.g., a surgical wound). In any embodiment, the first tissue-contact layer 158 can comprise a woven fabric (e.g., gauze) or a non-woven fabric-like material. FIGS. 9A and 9B show separate views of one embodiment of a second sheet 255 that comprises a plurality of layers. The plurality comprises a second substrate layer 257 and a second tissue-contact layer 258. In any embodiment, the second substrate layer 257 can comprise a polymeric film. Suitable films include multilayer films (e.g., coextruded films) and/or a film coated with a solvent solution polymer or polymer dispersion. Suitable polymers include polyolefins, polyesters, polyamides, polyurethanes, acrylic block and random copolymers, polyether amides, polyether polyesters, polyolefinpolyacrylate copolymers such as ethylenemethylacrylate, polyolefin copolymers with polar vinyl monomers such as polyethylene vinyl acetate and the like. The film preferably is formed from a transparent or translucent polymeric material. The material preferably permits moisture evaporation through the film during prolonged surgeries. Suitable materials include polyolefins, such as low density polyethylene and particularly metallocene polyethylenes such as ENGAGE™ polyethylenes commercially available from Dow Chemical, polyurethanes such as polyester or polyether polyurethanes (e.g., “ESTANE™ thermoplastic polyurethane,” commercially available from B. F. Goodrich, Cleveland Ohio), polyesters such as polyether polyester (e.g., “HYTREL™ polyester elastomer,” commercially available from Du Pont Co., Wilmington, Del.), and polyamides such as polyether polyamides (e.g., “PEBAX™ Resins” commercially available from ELF Atochem, North America, Inc., Philadelphia, Pa.).

The second substrate layer 257, when present, functions to provide the tensile strength necessary to retract tissue when the second sheet is urged against the tissue. In any embodiment, the second substrate layer 257 may comprise a light-guiding polymeric film such as, for example, the light-guiding films described in U.S. Pat. Nos. 9,070,312 and 9,039,245. In any embodiment, the second sheet 250 may comprise an optically-diffusing (e.g., light-scattering) polymeric film. Diffuse light can be provided, for example, by providing a texture on the wound-facing surface of the sheet. In any embodiment, the texture can be a random matte finish or an engineered microreplicated pattern. In these embodiments, it is preferable the side of the sheet that receives the light has a low refractive index. This can be provided by fluoropolymer films, for example.

The second tissue-contact layer 258, when present, provides a biocompatible surface for contacting tissue in a wound (e.g., a surgical wound). In any embodiment, the second tissue-contact layer 258 can comprise a woven fabric (e.g., gauze) or a non-woven fabric-like material.

In any embodiment of a tissue retractor that includes a first sheet and/or second sheet comprising a plurality of layers, the first tissue-contact layer and/or second tissue-contact layer may be coextensive with the corresponding substrate layer (as shown in FIGS. 9A and 9B). In any embodiment of a tissue retractor that includes a first sheet and/or second sheet comprising a plurality of layers, the first tissue-contact layer and/or second tissue-contact layer may not be coextensive with the corresponding substrate layer (as shown in FIGS. 8A and 8B, wherein the second tissue-contact layer 258 is not as wide as the second substrate layer 257). As shown in FIG. 9B sheet 255 may be a multi-laminate sheet structure with an intermediate layer 259 positioned between layers 257 and 258, wherein layer 259 may provide an additional beneficial property such as tensile strength.

In any embodiment of a tissue retractor according to the present disclosure wherein the first sheet and second sheet each comprises a plurality of layers as described herein, the first substrate layer and second substrate layer each may be fabricated from the same material or from different materials. In addition, the first tissue-contact layer and second tissue-contact layer each may be fabricated from the same material or from different materials. In any embodiment, the first tissue-contact layer and/or the second tissue contact layer can be a textured film surface, such as an engineered microreplicated pattern.

In any embodiment, the first sheet and or second sheet of a tissue retractor according to the present disclosure may be transmissible (e.g., substantially transmissible) with respect to electromagnetic radiation having a wavelength of about 200 nm to about 1100 nm. “Substantially transmissible”, as used herein means the first sheet or second sheet absorbs less than 50% of at least one wavelength of electromagnetic radiation within the aforementioned range of wavelengths. Using a light-transmissible material for the first and/or second sheets provides the ability to direct light through the sheet in order to visualize the tissue being retracted by the retractor and/or the open area created via the action of the tissue retractor. Preferred sheet materials are visually transparent to allow the clinician to observe the tissue that is contacted by the first sheet and/or second sheet of the retractor. Preferred materials include polymeric films that have a thickness of 0.5-20 mils (12-500 microns) and preferably 25-100 microns. First sheets or second sheets should have greater than 50% transmission through a single layer when measured at 550 nm. More preferably the first sheets or second sheets have greater than 60%, more preferably 70%, even more preferably 80% and most preferably greater than 85% transmission when measured at 550 nm. Most preferred sheets (e.g., polymeric films) have greater than 50% transmission across the entire visible spectrum from 400-750 nm

The electromagnetic radiation can be provided to the first sheet and/or second sheet via an external electromagnetic radiation source or, alternatively, the electromagnetic radiation can be provided to the first sheet and/or second sheet via a source of electromagnetic radiation that is optically coupled to the sheet(s). An example of a tissue retractor comprising a source of electromagnetic radiation is disclosed in U.S. Patent Application Publication No. 2012/0149992, which is incorporated herein by reference in its entirety.

In any embodiment, a tissue retractor according to the present disclosure optionally can comprise a grip structure, such as, for example, the engagement region 20 of the handle 12 of the device 10 shown in FIG. 7A of U.S. Patent Application Publication No. 2013/144959, which is incorporated herein by reference in its entirety.

The present disclosure further provides a method of retracting tissue at a wound (e.g., incision) site. The method comprises inserting through an opening in skin the second and fourth longitudinal members of any embodiment of a tissue retractor according to the present disclosure. After inserting the second and fourth longitudinal members through the skin, the second and fourth longitudinal members are positioned proximate one side (i.e., the internal side) of the skin and the first and third longitudinal members are positioned proximate the opposite side (i.e., the external side) of the skin. The method further comprises applying a force to urge the first sheet away from the second sheet or to urge the second sheet away from the first sheet. The first sheet can be urged away from the second sheet and/or the second sheet can be urged away from the first sheet, for example, using a translating element as described herein.

In any embodiment of the method, urging the first sheet away for the second sheet and/or urging the second sheet away from the first sheet comprises urging a first segment of skin away from a second segment of skin, wherein the first segment is in contact exclusively with the first sheet and wherein the second segment is in contact exclusively with the second sheet. In any embodiment, a segment of a sub-dermal tissue layer (e.g., a sub-dermal fatty tissue layer) may also be in contact exclusively with the first sheet or the second sheet.

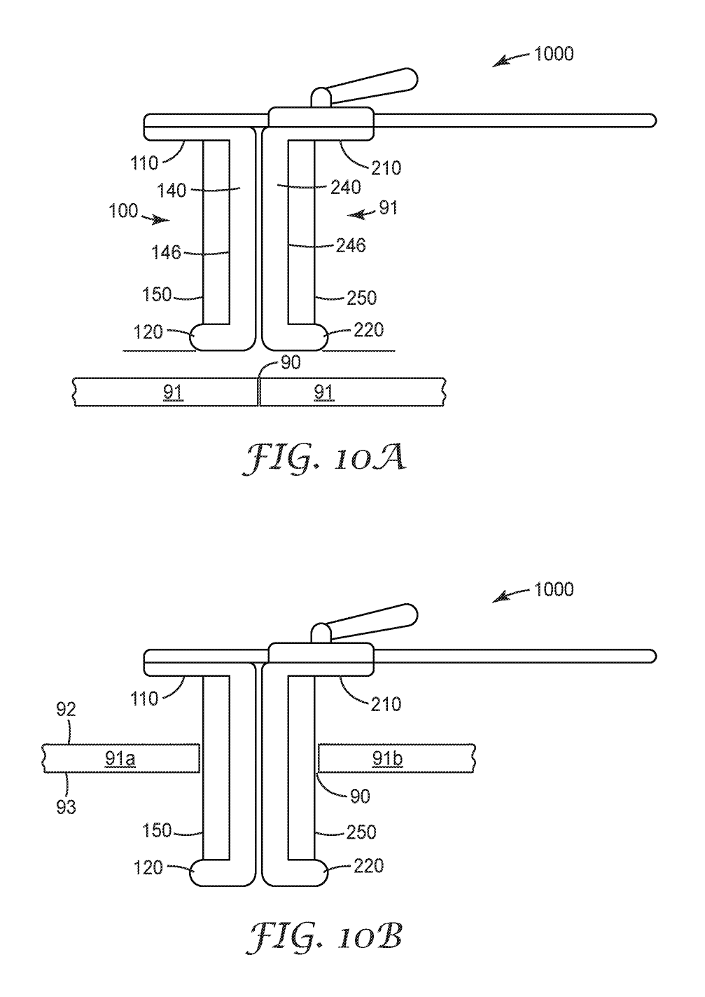

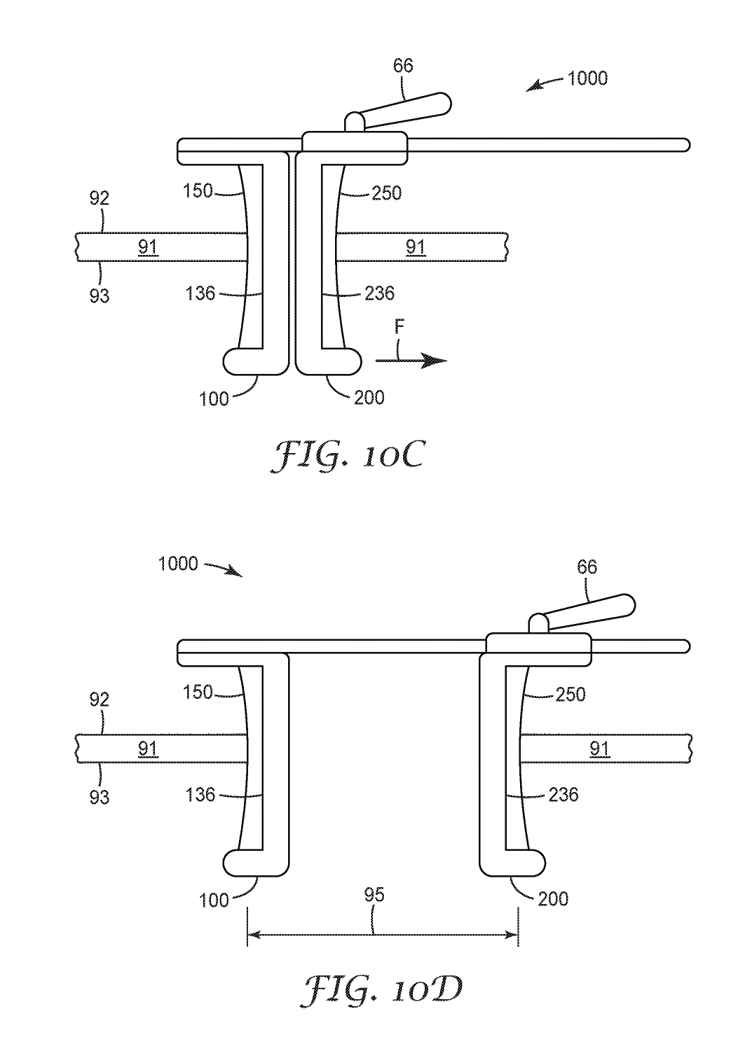

FIGS. 10A-D show a wound site (e.g., an incisional wound) at various steps in which the tissue retractor 1000 of FIGS. 1-5B is used to retract a tissue 91 (e.g., skin) surrounding the incisional wound. The tissue retractor 1000 used in the method can be any one of the embodiments disclosed herein. In the illustrated embodiment of FIG. 10A, the tissue retractor 1000 includes a first tissue-support assembly 100 that comprises inter alia a first longitudinal member 110, a second longitudinal member 120, and a second lateral member 140 comprising a second indent region 146 that extends between the first longitudinal member and the second longitudinal member. Also extending between the first longitudinal member 110 and the second longitudinal member 120 is a first sheet 150, as described herein. The first sheet 150 is spaced-apart from the second indent region 146. The tissue retractor 1000 further includes a second tissue-support assembly 200 that comprises inter alia a third longitudinal member 210, a fourth longitudinal member 220, and a fourth lateral member 240 comprising a fourth indent region 246 that extends between the third longitudinal member and the fourth longitudinal member. Also extending between the third longitudinal member 210 and the fourth longitudinal member 220 is a second sheet 250, as described herein. The second sheet 250 is spaced-apart from the fourth indent region 246.

As shown in FIG. 10A, before using the tissue retractor 1000 to retract edges of the wound, the tissue retractor is positioned proximate the incision site 90 in the tissue 91 that is to be retracted. The tissue retractor 1000 is prepared for use (i.e., placed into a first operational configuration) by positioning the first tissue-support assembly 100 adjacent the second tissue-support assembly 200 in order to facilitate insertion of portions (e.g., the second lateral member, fourth lateral member, the first sheet 150 and second sheet 250) of the tissue support assemblies into the wound at the incision site.

FIG. 10B shows the second lateral member 140 and the fourth lateral member 240 of the tissue retractor 1000 inserted into the incision site 90 such that the first longitudinal member 110 and third longitudinal member 210 of the tissue retractor are positioned proximate one side (i.e., the external side 92) of the tissue 91 (e.g., skin) and the second and fourth lateral members (140 and 240, respectively) are positioned proximate the other side (i.e., the internal side 93) of the tissue 91 at the incision site 90. In this position, a first segment 91a of tissue is in contact (e.g., exclusive contact) with the first sheet 150 and a second segment 91b of tissue is in contact (e.g., exclusive contact) with the second sheet 250.

FIG. 10C shows the tissue retractor 1000 and incision site 90 as the tissue retractor is moved into a second operational configuration. In this configuration, the second tissue-support assembly 200 is urged in a direction “F” away from the first tissue-support assembly. It is contemplated that, alternatively or additionally, the tissue retractor 1000 could be placed into the second operational configuration by applying a force to move the first tissue-support assembly 100 away from the second tissue-support assembly (i.e., in a direction opposite “F”, not shown). In this step, portions of the tissue 91 (and, optionally, underlying tissue such as subdermal fat, for example) is in contact with (e.g., exclusive contact) and is subject to force applied by the first sheet 150 and second sheet 250, respectfully. The force can be applied, for example, using the translating element 66 (e.g., crank handle). Although the first and or second sheet (first sheet 150 and second sheet 250, respectively) may flex due to the resistive force of the tissue 91, neither the first sheet nor the second sheet contact one of the indent regions (second indent region 146 and fourth indent region 246, respectively)

FIG. 10D shows the tissue retractor 1000 and incision site 90 after the tissue retractor 1000 has been placed into a third operational configuration. In this operational configuration, the first sheet 150 and second sheet 250 have been urged further apart in order to further open the wound site for treatment. This results in the creation of a substantial gap 95 that allows medical personnel to expose (i.e., provide visual and operational access to) the tissue and/or organs beneath (e.g., directly beneath) the original incision site.

### EXEMPLARY EMBODIMENTS

Embodiment A is a tissue retractor, comprising:

a first tissue-support assembly comprising:
a first longitudinal member having a first longitudinal end, a second longitudinal end opposite the first longitudinal end, and a first edge;
a second longitudinal member having a third longitudinal end, a fourth longitudinal end opposite the third longitudinal end, and a second edge;
a first lateral member extending between the first and second longitudinal members, the first lateral member having a first upper end attached to the first longitudinal member, a first lower end attached to the second longitudinal member, and a first indent region disposed between the first upper end and the first lower end;
a first sheet of pliable tissue-contact material coupled to the first tissue-support assembly, the first sheet extending from the first edge to the second edge;

a second tissue-support assembly coupled to the first tissue-support assembly, the second tissue-support assembly comprising:
a third longitudinal member having a fifth longitudinal end, a sixth longitudinal end opposite the fifth longitudinal end, and a third edge;
a fourth longitudinal member having a seventh longitudinal end, an eighth longitudinal end opposite the seventh longitudinal end, and a fourth edge;
a second lateral member extending between the third and fourth longitudinal members, the second lateral member having a second upper end attached to the third longitudinal member, a second lower end attached to the fourth longitudinal member, and a second indent region disposed between the second upper end and the second lower end;
a second sheet of pliable tissue-contact material coupled to the second tissue-support assembly, the second sheet extending from the third edge to the fourth edge;

wherein the first sheet comprises a first face and a second face opposite the first face, wherein the first face faces away from the first indent region;

wherein the second sheet comprises a third face and a fourth face opposite the third face, wherein the third face faces away from the third indent region;

wherein the first face faces away from the third face;

wherein the first sheet does not contact the first lateral member;

wherein the second sheet does not contact the second lateral member.

Embodiment B is the tissue retractor of Embodiment A, wherein the first upper end is attached to the first longitudinal member proximate the first longitudinal end.

Embodiment C is the tissue retractor of Embodiment A or Embodiment B, wherein the first lower end is attached to the second longitudinal member proximate the third longitudinal end.

Embodiment D is the tissue retractor of any one of the preceding Embodiments, wherein the second upper end is attached to the third longitudinal member proximate the fifth longitudinal end.

Embodiment E is the tissue retractor of any one of the preceding Embodiments, wherein the second lower end attached to the fourth longitudinal member proximate the seventh longitudinal end.

Embodiment F is the tissue retractor of any one of the preceding Embodiments, wherein the first sheet does not contact the first indent region.

Embodiment G is the tissue retractor of any one of the preceding Embodiments, wherein the second sheet does not contact the second indent region.

Embodiment H is the tissue retractor of any one of the preceding Embodiments, wherein the first tissue-support assembly further comprises a second lateral member extending between the first and second longitudinal members;

wherein the second lateral member has a second upper end attached to the first longitudinal member, a second lower end attached to the second longitudinal member, and a second indent region disposed between the second upper end and the second lower end;

wherein the second lateral member is spaced apart from the first lateral member;

wherein the first sheet does not contact the second lateral member.

Embodiment I is the tissue retractor of Embodiment G, wherein the second upper end is attached to the first longitudinal member proximate the second longitudinal end.

Embodiment J is the tissue retractor of Embodiment H or Embodiment I, wherein the second lower end is attached to the second longitudinal member proximate the fourth longitudinal end.

Embodiment K is the tissue retractor of any one of Embodiments H through J, wherein the first sheet does not contact the second indent region.

Embodiment L is the tissue retractor of any one of the preceding Embodiments, wherein the second tissue-support assembly further comprises a fourth lateral member extending between the third and fourth longitudinal members;

wherein the fourth lateral member has a fourth upper end attached to the third longitudinal member, a fourth lower end attached to the fourth longitudinal member, and a fourth indent region disposed between the fourth upper end and the fourth lower end;

wherein the fourth lateral member is spaced apart from the third lateral member;

wherein the second sheet does not contact the fourth lateral member.

Embodiment M is the tissue retractor of Embodiment L, wherein the fourth upper end is attached to the third longitudinal member proximate the sixth longitudinal end.

Embodiment N is the tissue retractor of Embodiment L or Embodiment M, wherein the fourth lower end is attached to the fourth longitudinal member proximate the eighth longitudinal end.

Embodiment O is the tissue retractor of any one of Embodiments L through N, wherein the second sheet does not contact the fourth indent region.

Embodiment P is the tissue retractor of any one of the preceding Embodiments, wherein the second tissue-support assembly is movably coupled to the first tissue-support assembly.

Embodiment Q is the tissue retractor of Embodiment P, further comprising a position controller that fixes the position of the first tissue-support assembly relative to the second tissue-support assembly.

Embodiment R is the tissue retractor of any one of the preceding Embodiments, wherein the first sheet comprises at least one first fenestration extending therethrough.

Embodiment S is the tissue retractor of Embodiment R, wherein the at least one fenestration extending through the second sheet extends from the first edge to the second edge.

Embodiment T is the tissue retractor of any one of the preceding Embodiments, wherein the second sheet comprises at least one fenestration extending therethrough.

Embodiment U is the tissue retractor of Embodiment T, wherein the at least one fenestration extending through the second sheet extends from the third edge to the fourth edge.

Embodiment V is the tissue retractor of any one of the preceding Embodiments, wherein the first sheet comprises a first portion that overlaps a part of the first longitudinal member, a second portion that overlaps a part of the second longitudinal member, and a third portion that extends between the first portion and second portion and does not overlap either a part of the first longitudinal member or a part of the second longitudinal member; wherein the third portion forms a continuous flat plane.

Embodiment W is the tissue retractor of any one of Embodiments A through U, wherein the first sheet comprises a first portion that overlaps a part of the first longitudinal member, a second portion that overlaps a part of the second longitudinal member, and a third portion that extends between the first portion and second portion and does not overlap either a part of the first longitudinal member or a part of the second longitudinal member; wherein the third portion forms a continuous curved plane.

Embodiment X is the tissue retractor of any one of the preceding Embodiments, wherein the second sheet comprises a fourth portion in contact with the third longitudinal member, a fifth portion that overlaps a part of the fourth longitudinal member, and a sixth portion that extends between the third longitudinal member and the fourth longitudinal member and does not overlap either a part of the third longitudinal member or a part of the fourth longitudinal member; wherein the sixth portion forms a continuous flat plane.

Embodiment Y is the tissue retractor of any one of Embodiments A through U, wherein the second sheet comprises a fourth portion that overlaps a part of the third longitudinal member, a fifth portion that overlaps a part of the fourth longitudinal member, and a sixth portion that extends between the third longitudinal member and the fourth longitudinal member and does not overlap either a part of the third longitudinal member or a part of the fourth longitudinal member; wherein the sixth portion forms a continuous curved plane.

Embodiment Z is the tissue retractor of any one of the preceding Embodiments, further comprising a translating element that is adapted to move the first tissue-support assembly away from the second tissue-support assembly or to move the second tissue-support assembly away from the first tissue-support assembly.

Embodiment AA is the tissue retractor of Embodiment Z, wherein the translating element is adapted to move the first tissue-support assembly toward the second tissue-support assembly or to move the second tissue-support assembly toward the first tissue-support assembly.

Embodiment AB is the tissue retractor of any one of the preceding Embodiments, wherein the first sheet or the second sheet comprises a plurality of layers.

Embodiment AC is the tissue retractor of Embodiment AB, wherein the plurality of layers comprises a tissue-contact layer and a substrate layer.

Embodiment AD is the tissue retractor of any one of the preceding Embodiments, wherein the first sheet, second sheet, or a layer of the first sheet or second sheet comprises a polymeric film.

Embodiment AE is the tissue retractor of any one of the preceding Embodiments, wherein the first sheet, second sheet, or a layer of the first sheet or second sheet comprises a woven fabric or nonwoven fabric-like material.

Embodiment AF is the tissue retractor of any one of the preceding Embodiments, wherein the first sheet has a first strain limit of about 2 MPa to about 2 GPa.

Embodiment AG is the tissue retractor of any one of the preceding Embodiments, wherein the second sheet has a second strain limit of about 2 MPa to about 2 GPa.

Embodiment AH is the tissue retractor of any one of the preceding Embodiments, wherein at least one of the longitudinal members or lateral members comprises a metal, a thermoplastic polymeric material, or a combination thereof.

Embodiment AI is the tissue retractor of any one of the preceding Embodiments, wherein first sheet or second sheet is transmissible with respect to an electromagnetic radiation having a wavelength of about 200 nm to about 1100 nm.

Embodiment AJ is the tissue retractor of Embodiment AI, wherein the tissue retractor further comprises a source of the electromagnetic radiation operatively coupled thereto, wherein the sheet that is transmissible with respect to the electromagnetic radiation is optically coupled to the source of the electromagnetic radiation.

Embodiment AK is the tissue retractor of any one of the preceding Embodiments, wherein the first sheet, the second sheet, or a layer of the first sheet or second sheet is configured for light scattering or light orienting.

Embodiment AL is the tissue retractor of any one of the preceding Embodiments, wherein the first tissue-support assembly or second tissue-support assembly further comprises a grip structure.

Embodiment AM is a method, comprising:

inserting the second and fourth longitudinal members of a tissue retractor of any one of the preceding Embodiments through an opening in a portion of skin of a body;
wherein the skin has an internal side and an external side;
wherein inserting the second and fourth longitudinal members comprises positioning the tissue retractor such that the second and fourth longitudinal members are disposed proximate the internal side of the skin and the first and third longitudinal members are disposed proximate the external side of the skin; and

applying a force to urge the first sheet away from the second sheet or to urge the second sheet away from the first sheet.

Embodiment AN is a method of retracting an organ, the method comprising:

inserting the first sheet and second sheet of a tissue retractor of any one of the preceding claims through an opening in a portion of skin of a body;
wherein inserting the second and fourth longitudinal members comprises positioning the tissue retractor such that the first sheet or second contacts an organ; and

applying a force to urge the first sheet away from the second sheet or to urge the second sheet away from the first sheet.

The complete disclosure of all patents, patent applications, and publications, and electronically available material cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A tissue retractor, comprising:
a first tissue-support assembly movably coupled to a second tissue-support assembly; wherein the first tissue-support assembly comprises:
a first longitudinal member having a first longitudinal end, a second longitudinal end opposite the first longitudinal end, and a first edge;
a second longitudinal member having a third longitudinal end, a fourth longitudinal end opposite the third longitudinal end, and a second edge;
a first lateral member extending between the first and second longitudinal members, the first lateral member having a first upper end attached to the first longitudinal member, a first lower end attached to the second longitudinal member, and a first indent region disposed between the first upper end and the first lower end;
a first sheet of pliable tissue-contact material coupled to the first tissue-support assembly, the first sheet extending from the first edge to the second edge;
a second tissue-support assembly coupled to the first tissue-support assembly, the second tissue-support assembly comprising:
a third longitudinal member having a fifth longitudinal end, a sixth longitudinal end opposite the fifth longitudinal end, and a third edge;
a fourth longitudinal member having a seventh longitudinal end, an eighth longitudinal end opposite the seventh longitudinal end, and a fourth edge;
a third lateral member extending between the third and fourth longitudinal members, the third lateral member having a third upper end attached to the third longitudinal member, a third lower end attached to the fourth longitudinal member, and a third indent region disposed between the third upper end and the third lower end;
wherein the first sheet comprises a first face and a second face opposite the first face, wherein the first face faces away from the first indent region;
wherein the second sheet comprises a third face and a fourth face opposite the third face, wherein the third face faces away from the third indent region;
wherein the first face faces away from the third face;
wherein the first face faces away from the second face;
wherein the first sheet does not contact the first lateral member;
wherein the second sheet does not contact the third lateral member.

2. The tissue retractor of claim 1, wherein the first upper end is attached to the first longitudinal member proximate the first longitudinal end.

3. The tissue retractor of claim 1, wherein the first lower end is attached to the second longitudinal member proximate the third longitudinal end.

4. The tissue retractor of claim 1, wherein the first sheet does not contact the first indent region.

5. The tissue retractor of claim 1, wherein the first tissue-support assembly further comprises a second lateral member extending between the first and second longitudinal members;
wherein the second lateral member has a second upper end attached to the first longitudinal member, a second lower end attached to the second longitudinal member, and a second indent region disposed between the second upper end and the second lower end;
wherein the second lateral member is spaced apart from the first lateral member;
wherein the first sheet does not contact the second lateral member.

6. The tissue retractor of claim 5, wherein the first sheet does not contact the second indent region.

7. The tissue retractor of claim 1, wherein the second tissue-support assembly further comprises a fourth lateral member extending between the third and fourth longitudinal members;
wherein the fourth lateral member has a fourth upper end attached to the third longitudinal member, a fourth lower end attached to the fourth longitudinal member, and a fourth indent region disposed between the fourth upper end and the fourth lower end;
wherein the second sheet does not contact the fourth lateral member.

8. The tissue retractor of claim 7, wherein the fourth upper end is attached to the third longitudinal member proximate the sixth longitudinal end.

9. The tissue retractor of claim 7, wherein the fourth lower end is attached to the fourth longitudinal member proximate the eighth longitudinal end.

10. The tissue retractor of claim 1, wherein the second tissue-support assembly is moveably coupled to the first tissue-support assembly.

11. The tissue retractor of claim 10, further comprising a position controller that fixes the position of the first tissue-support assembly relative to the second tissue-support assembly.

12. The tissue retractor of claim 1, wherein the first sheet comprises at least one first fenestration extending therethrough.

13. The tissue retractor of claim 1, further comprising a translating element that is adapted to move the first tissue-support assembly away from the second tissue-support assembly or to move the second tissue-support assembly away from the first tissue-support assembly.

14. The tissue retractor of claim 1, wherein the first sheet or the second sheet comprises a plurality of layers.

15. The tissue retractor of claim 1, wherein the first sheet has a first strain limit of about 2 MPa to about 2 GPa.

16. The tissue retractor of claim 1, wherein at least one of the longitudinal members or lateral members comprises a metal, a thermoplastic polymeric material, or a combination thereof.

17. The tissue retractor of claim 1, wherein first sheet or second sheet is transmissible with respect to an electromagnetic radiation having a wavelength of about 200 nm to about 1100 nm.

18. The tissue retractor of claim 1, wherein the first sheet, the second sheet, or a layer of the first sheet or second sheet is configured for light scattering or light orienting.

19. A method of retracting tissue at an incision site, the method comprising:
inserting the second and fourth longitudinal members of a tissue retractor of claim 1 through an opening in a portion of skin of a body;
wherein the skin has an internal side and an external side;
wherein inserting the second and fourth longitudinal members comprises positioning the tissue retractor such that the second and fourth longitudinal members are disposed proximate the internal side of the skin and the first and third longitudinal members are disposed proximate the external side of the skin; and
applying a force to urge the first sheet away from the second sheet or to urge the second sheet away from the first sheet.

20. A method of retracting an organ, the method comprising:
inserting the second and fourth longitudinal members of a tissue retractor of claim 1 through an opening in a portion of skin of a body;
wherein the skin has an internal side and an external side;
wherein inserting the second and fourth longitudinal members comprises positioning the tissue retractor such that the first sheet or second contacts an organ proximate the internal side of the skin and the first and third longitudinal members are disposed proximate the external side of the skin; and
applying a force to urge the first sheet away from the second sheet or to urge the second sheet away from the first sheet.
